(19) United States
(12) Patent Application Publication
(10) Pub. No.: US 2002/0071786 A1
(43) Pub. Date: Jun. 13, 2002

(54) Continuous emissions monitor for measuring organic constituents

(76) Inventors: Robert J. Schreiber JR., Fenton, MO (US); Brad Phillips, Eureka, MO (US)

Correspondence Address: JOHN S. PRATT, KILPATRICK STOCKTON LLP, 1100 PEACHTREE, SUITE 2800, ATLANTA, GA, 30309, US

(21) Appl. No.: 09/731,883

(22) Filed: Dec. 7, 2000

(51) Int. Cl. G01N 1/00; G01N 33/00

(52) U.S. Cl. 422/83; 436/174; 436/177; 422/89

(57) ABSTRACT

The present invention comprises a system and method for continuously monitoring a discharge stream emission to determine the amount of toxins or pollutants, such as certain organic compounds, within the emission. The system and method are temperature controlled to ensure that the toxins or pollutants are neither formed nor destroyed in the process of obtaining a sample for analysis. The system and method use a temperature-controlled sampling means, means for removing the toxins or pollutants, water removal means, and means for determining the concentration of the toxins or pollutants in the sample, and therefore the emission. The present invention may also include means for removing particulate materials from the sample.

### FIELD OF THE INVENTION

This invention relates to a system and method for monitoring an emission to measure the amount of toxins or pollutants of interest located within the emission. The system and method are able to continuously monitor the emission over varied lengths of time while ensuring that the toxins or pollutants of interest are neither formed nor destroyed in the process of obtaining a sample for analysis.

### BACKGROUND OF THE INVENTION

Environmental concerns related to the discharge of liquid and gaseous waste into the environment have grown in importance over the last few years. The Environmental Protection Agency (EPA) has promulgated guidelines and regulations related to these discharges requiring that the discharges meet basic maximums for the amounts of different toxins and pollutants which may be accompany a discharge. Over the years, the EPA has continued to lower these maximums as technology has improved in dealing with the removal of these toxins and pollutants from the various discharge streams. Depending on the toxin or pollutant of interest, these levels may be in the range of micrograms, nanograms, or even picograms per liter. However, while the technology for removing these toxins and pollutants from a discharge have improved, the technology related to measuring the residual amounts in the discharge has not kept pace. Accordingly, it is increasingly difficult to measure the discharge stream to determine whether the EPA guidelines related to that discharge are being satisfied.

Technology does exist for determining the amount of a particular toxin or pollutant in a discharge stream down to the picogram/liter level, or even lower. However, this technology is complex and is therefore usually located within a laboratory or other testing facility. Therefore, for some discharge streams, samples are taken from the stream, sent to the lab or testing facility and analyzed. However, this method provides only semi-periodic testing of the discharge stream, and if the composition of the stream should change between testing periods, could result in higher levels of the contaminant being released into the environment thereby damaging the surrounding ecology.

Additionally, some testing procedures do not take into account the temperature of the discharge stream when obtaining the sample, especially when the discharge stream is a gaseous stream. If the sample is taken at too low a temperature, excess water in the stream may condense prior to testing the sample, which could interfere with the results. Additionally, additional reaction products might result, increasing the level of the toxin or pollutant in the sample to a level above the actual level of the toxin or pollutant in the discharge stream. Conversely, if the sample is taken at too high a temperature, some of the toxin or pollutant in the sample may be destroyed, thereby decreasing the level of the toxin or pollutant in the sample to a level below the actual level of the toxin or pollutant in the discharge stream. Even if the sample is taken at a proper temperature, these same problems might also arise if the sample is not maintained within a desired temperature range prior to testing the sample.

Accordingly, there is a need for a monitoring system and method which is able to overcome these problems with the current testing systems. There is a need for a monitoring system which is capable of continuously monitoring a discharge stream emission over varied lengths of time to ensure that the discharge stream emission is satisfying EPA guidelines related to that discharge stream emission. Additionally, there is a need for a monitoring system which controls the temperature of the system to ensure that the toxins or pollutants of interest are neither formed nor destroyed in the process of obtaining a sample for analysis.

### SUMMARY OF THE INVENTION

The present invention satisfies these needs by providing a system and method for continuously monitoring the concentration of toxins and pollutants, such as certain organic constituents, within an emission. The present invention also provides a system and method for continuously monitoring the concentration of toxins and pollutants which is temperature controlled. The present invention is an effective alternative to prior art methods which only periodically test the emission and do not take into account the temperature of the sample or the emission stream.

The present invention is designed to perform source emissions monitoring over varied lengths of time from relatively short emissions testing to long-term. Primarily the system is designed to be used as a continuous monitoring of a source for certain target organic compounds. These organic compounds, include, but are not limited to, polychlorinated dibenzodioxin/polychlorinated dibenzo furans (PCDD/PCDF), polycyclic aromatic hydrocarbons (PAH) and polychlorinated biphenyls (PCB).

The present invention includes a sampling means, means for removing the organic constituent of interest, water removal means, and means for determining the concentration of the organic constituent of interest in the sample, and therefore the emission. The present invention may also include means for removing particulate materials from the sample. Preferably, the entire system is temperature controlled.

The present invention thus provides an inexpensive and easy method of continuously monitoring the concentration of certain organic constituents within an emission.

Accordingly, it is an object of the present invention to provide a system for continuously monitoring the concentration of certain organic constituents within an emission.

It is another object of the present invention to provide a method for continuously monitoring the concentration of certain organic constituents within an emission.

It is a further object of the invention to provide a temperature-controlled system for continuously monitoring the concentration of certain organic constituents within an emission.

It is a still further object of the invention to provide a temperature-controlled method for continuously monitoring the concentration of certain organic constituents within an emission.

These and other objects of the invention will be apparent to those skilled in the art from the following description of the preferred embodiment thereof in association with the accompanying drawing.

### BRIEF DESCRIPTION OF THE DRAWING

FIG. 1 is a diagram of one embodiment of the continuous emissions monitor of the present invention.

### DETAILED DESCRIPTION

The present invention is directed to a system and method for monitoring emissions for organic constituents. The system and method are able to continuously monitor the emission to determine the level of the organic constituents in the emission to help regulate the discharge of the emission such that environmental concerns are satisfied.

The system and method comprise an improvement in the prior art sampling systems. Typically, an emission was checked by taking a sample or plurality of samples over a period of time, and then sending the samples to be analyzed. However, this non-continuous process is not very accurate as the composition of the emission may change over time and the periodic samples may not reflect this changing composition.

However, the present invention uses a system which is capable of continuously monitoring the emission stream. By continuously measuring the emission, any changes in the composition of the emission may be determined, and subsequent changes to the overall process may be implemented as necessary.

The present invention is able to continuously monitor the emission by utilizing a system having a sampling means located within the emission stream. Typically, this emission stream may be located within a gaseous stream stack. Next, after the sample is pulled from the stack, the sample is sent to the next part of the system wherein the organic constituent or constituents of interest are removed. Then any residual water from the sample is also removed to form a dry gas sample having no water or organic constituents of interest. By measuring the flow rate of the dry gas in relation to the initial flow rate of the sample and the amount of water removed, it is possible to determine the concentration of the organic constituents of interest within the sample stream. Since the sample stream is pulled directly from the emission stream, it comprises a representative sample of the emission. Therefore, it is then possible to determine the overall amount of the organic constituents of interest in the emission stream. If the level is too high, changes to the overall process may be implemented to help satisfy any environmental concerns related to the organic constituents of interest.

One key aspect of the present invention is the sampling probe. Unlike many sampling probes used in the prior art, the present invention uses a temperature-controlled sampling probe. Since most stacks are heated, the emission, and therefore any sample, are also heated. If a sample is pulled using a probe that is either too cold or too hot, the sudden change in temperature may adversely affect any subsequent readings and interfere with obtaining an accurate measurement of the composition of the emission stream. However, by controlling the temperature of the sample probe, it is possible to prevent these problems.

Accordingly, the present invention uses a temperature-controlled sampling probe. The probe is preferably controlled at a temperature above the dew point of the sample, which is typically around 230° F. to 250° F. However, depending on the amount of water in the sample, the dew point may be as low as 160° F., or even lower. By using a temperature above the dew point, any water in the sample remains in a gaseous state until it is removed, therefore ensuring an accurate determination of the composition of the sample. However, if the sampling probe is maintained at too high a temperature, then other problems may arise, such as destruction of the target organic compounds, again adversely affecting the determination of the sample composition. Accordingly, it is preferred that the sampling probe be maintained at a temperature less than about 400° F.

The temperature of the sampling probe may be controlled using any known type of temperature control including, but not limited to, heat exchangers, water, or refrigerant. Since the stack emission will typically be above about 400° F., the temperature control means will typically involve a cooling-type of system. Preferably, the temperature is controlled using a water-cooling system, since these types of systems are relatively inexpensive to build and operate. However, as previously discussed, any known temperature control means may be used, depending on the operating parameters of the system.

After the sample is pulled, it is preferably filtered to remove any particulate material. Then, the sample is sent to an organic trap which removes the organic constituent or constituents of interest. The organic trap may comprise any known means for removing an organic material. The type of trap used will depend on the type of organic compound to be removed and its concentration in the sample. Typically, the organic trap will comprise a resin material to which the organic constituent or constituents of interest will be removed from the sample. The resin may comprise either an adsorbent or an absorbent resin, depending on the constituent or constituents of interest. Alternatively, a condenser may be used if the concentration of the organics in the sample is sufficiently low.

After the organics have been removed, the first treated sample is then treated to remove any water from the sample to produce a dry gas sample. As will be discussed hereafter, any of a number of different water removal means may be used, such as condensers and silica-gel systems. The amount of water removed from the sample is then measured to help determine the level of organic constituents removed.

Preferably, the entire system and method, including the organic trap and the water removal system, are temperature controlled to ensure that the target organics are neither formed nor destroyed in the process of obtaining a sample for analysis.

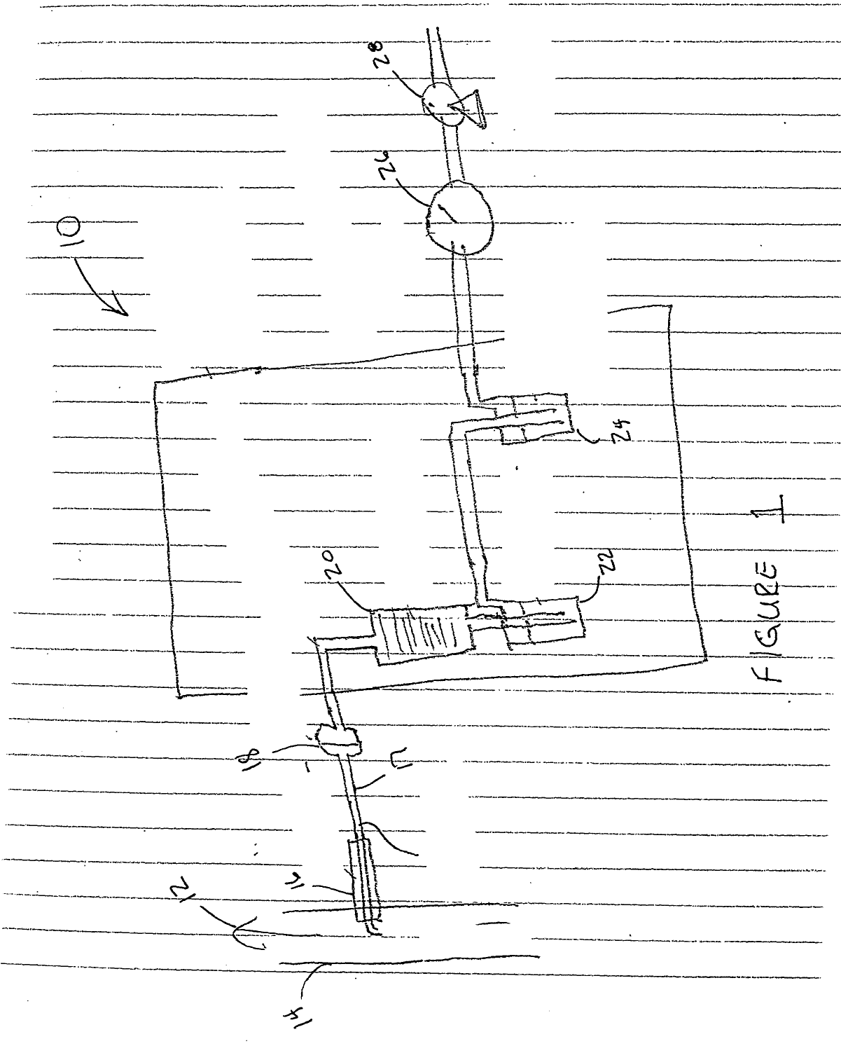

Referring now to FIG. 1, there is depicted one embodiment of the present invention. The system 10 is used to monitor the amount of an organic constituent or constituents in an emission 12 from a stack 14. The system 10 includes a temperature-controlled sampling probe unit 16 and a sample line 17. The sampling probe 16 is designed to extract a sample from the emission source 12 to be measured and direct that sample into the system's 10 sampling train. The probe 16 is preferably constructed of suitable materials based on the composition of the source emissions. Example materials of construction include, but are not limited to, titanium, stainless steel, glass, and TEFLON®. The sampling probe 16 may be designed to sample isokinetically or non-isokinetically to obtain a representative sample for particulate. As discussed, the sampling probe can be either heated or cooled depending on the source temperature profile. The sample probe unit 16 may also include a filter (not shown) within the source sample extraction point. The sample probe 16 may also include temperature sensors and control systems to monitor and maintain the unit at the predetermined temperature level.

After the probe unit 16 pulls the sample, the sample line 17 directs the sample from the probe unit 16 to a filter system 18. The sample line 17 may also be heated or cooled, depending on the source temperature of the sample. The sample line 17 is preferably constructed of suitable materials based on the composition of the source emission and these materials may include, but are not limited to, stainless steel and TEFLON®. The sample line 17 may also be insulated with temperature sensors and control systems to monitor and maintain the predetermined temperature level.

Optionally, a filter system 18 may be used to remove any particulate material from the emission 12 which might interfere with the subsequent features of the system 10. The filter system 18 is preferably designed to remove particulate matter from the sample stream to prevent fouling of a resin system 20. The filter system 18 includes a housing which is preferably constructed of suitable materials based on the composition of the emissions source and can include, but are not limited to, glass, titanium and stainless steel.

The filter media (not shown) will also be based on the source emission composition and can include, but are not limited to, quartz wool, stainless mesh, or combinations thereof.

The filter system 18 is preferably heated or cooled, depending on the temperature of the sample, to maintain a preset temperature and may include appropriate sensors and controls to assure conformance to the desired sample temperature.

After passing through the filter system 18, the sample is sent to the resin system 20 for removing the organic constituent or constituents to produce a first treated sample. As discussed, the resin system 20 may comprise an adsorbent resin, absorbent resin, or condensation system, depending on the type and concentration of the organic constituent or constituents in the emission. However, the resin system 20 will likely comprise either an adsorbent resin or an absorbent resin.

The resin system 20 is designed to house a varied amount of absorptive resin or adsorptive resin, depending on the organic constituents to be removed. The resin is selected such that it is capable of capturing the pollutant of concern for the predetermined testing timeframe. The amount of resin utilized varies depending on the type of resin, source pollutant concentration and length of sampling. The resin system 20 may be heated or cooled, depending on the sample temperature received, and may contain any necessary sensors and controls to monitor and maintain the sample temperature at a predetermined value. Preferably, this temperature is between about 30° F. and about 40° F. to help ensure complete removal of any organic constituents. The resin system 10 includes a housing which is preferably constructed of appropriate materials based on the composition of the emission source sample. These materials may include, but are not limited to, glass and stainless steel.

After passing through the resin system 20, the first treated sample is sent to a moisture removal system. The moisture removal system is used to remove any water from the first treated sample to produce a dry gas sample. The moisture removal system may utilize any of numerous types of moisture removal means including, but not limited to, condensers 22 and silica-gel drying columns 24. The function of the moisture removal system is to remove the water content in order to determine dry gas flow. Examples of condensers 22 useful in the present invention could include standard refrigeration-type cold water exchangers. Any residual water may be removed using the silica-gel drying column 24. The condenser 22 and the silica-gel drying column 24 are also preferably maintained at a temperature between about 30° F. and about 40° F. to help ensure complete removal of any water from the sample.

Next, the dry gas sample is passed through a dry gas meter 26 to determine the amount of the dry gas sample. The purpose of the dry gas meter 26 is to measure the flow rate of sample through the sampling system 10. The system 10 may utilize any of numerous types of gas flow meters and may also include temperature and pressure sensors (not shown) for correlation of the flow rates to standard conditions. The system 10 may also include additional continuous emissions monitors placed after the sample train. These units may be used for a variety of reasons including, but not limited to, using an oxygen meter to correct the emission value to a 7% oxygen content.

A vacuum pump 28 may be used to obtain the sample and pull the sample through the system 10. The pump 28 is used to pull a certain amount of gas volume through the sampling system 10. This pump 28 is generally a standard vacuum pump, but various other types of suction devices may be employed depending on flow needs.

A control system (not shown) may be used to determine the amount of any organic constituent or constituents located within the emission. By knowing the volumetric flow rate of the dry gas sample, the volumetric flow rate of the original sample, and the amount of water removed, it is possible to determine the amount of organic constituent removed. Then, it is possible to correlate the amount of the organic constituent within the overall emission since the sample comprises a representative sample of the overall emission stream. The control system may either be a manual system, if short testing protocols are used, or an automated system, which could use programmable logic controllers, data loggers or other computer systems. The data logging systems may also provide computations to correlate data to standard conditions or correct for water vapor, depending on the operating parameters of the emission being tested.

The system and method of the present invention may be used in a number of different environments and may be used to monitor a number of different organic pollutants. For example, the system and method may be with cement kilns which burn environmental waste to monitor organic compounds, including, but not limited to, polychlorinated dibenzodioxin/polychlorinated dibenzo furans (PCDD/PCDF), polycyclic aromatic hydrocarbons (PAH) and polychlorinated biphenyls (PCB).

It should be understood, of course, that the foregoing relates only to preferred embodiments of the present invention and that numerous modifications or alterations may be made therein without departing from the spirit and the scope of the invention as set forth in the appended claims.

We claim:

1. A method of continuously monitoring an emission for an organic constituent comprising:
obtaining a sample from the emission using a temperature-controlled sampling means;
passing the sample through means for removing the organic constituent to produce a first treated sample;
passing the first treated sample through means for removing water from the first treated sample to produce a dry gas sample; and
determining the amount of the organic constituent removed;
wherein the means for removing the organic constituent and the means for removing water are temperature controlled.

2. The method of claim 1, wherein the temperature-controlled sampling means comprises a sampling probe having means for controlling the temperature of the sample.

3. The method of claim 1, wherein the temperature-controlled sampling probe is maintained at a temperature between a dew point for the sample and about 400° F.

4. The method of claim 1, wherein the means for removing the organic constituent comprises a resin capable of removing the organic constituent.

5. The method of claim 1, wherein the means for removing water comprises a condenser.

6. The method of claim 5, wherein the means for removing water further comprises a silica-gel moisture removal means.

7. The method of claim 1, wherein the amount of the organic constituent removed is determined by:
measuring a volumetric flow rate of the sample obtained;
measuring a volumetric flow rate of the dry gas sample;
measuring a volumetric flow rate of the water removed; and
determining the amount of organic constituent removed to determine a concentration of the organic constituent in the emission.

8. The method of claim 1, wherein the means for removing the organic constituent and the means for removing water are controlled at a temperature between about 30° F. to about 40° F.

9. A system for continuously monitoring an emission for an organic constituent comprising:
a temperature-controlled sampling means for obtaining a sample;
means for removing the organic constituent to produce a first treated sample;
means for removing water from the first treated sample to produce a dry gas sample; and
means for determining the amount of the organic constituent removed;
wherein the means for removing the organic constituent and the means for removing water are temperature controlled.

10. The system of claim 9, wherein the temperature-controlled sampling means comprises a sampling probe having means for controlling the temperature of the sample.

11. The system of claim 9, wherein the temperature-controlled sampling probe is capable of being maintained at a temperature between a dew point for the sample and about 400° F.

12. The system of claim 9, wherein the means for removing the organic constituent comprises a resin capable of removing the organic constituent.

13. The system of claim 9, wherein the means for removing water comprises a condenser.

14. The system of claim 13, wherein the means for removing water further comprises a silica-gel moisture removal means.

15. The system of claim 1, wherein the means for determining the amount of the organic constituent removed comprises:
means for measuring a volumetric flow rate of the sample obtained;
means for measuring a volumetric flow rate of the dry gas sample;
means for measuring a volumetric flow rate of the water removed; and
means for determining the amount of organic constituent removed to determine a concentration of the organic constituent in the emission.

16. The system of claim 9, wherein the means for removing the organic constituent and the means for removing water are controlled at a temperature between about 30° F. to about 40° F.
